(19) United States
(12) Patent Application Publication
(10) Pub. No.: US 2009/0105584 A1
(43) Pub. Date: Apr. 23, 2009

(54) FIDUCIAL MARKER DEPLOYMENT SYSTEM USING SINGLE STICK NEEEDLE AND METHOD OF USE

(75) Inventor: Andrew Jones, Lake Steven, WA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: CIVCO MEDICAL INSTRUMENTS CO., INC., Kalona, IA (US)

(21) Appl. No.: 12/211,436

(22) Filed: Sep. 16, 2008

Related U.S. Application Data
(60) Provisional application No. 60/980,926, filed on Oct. 18, 2007.

(51) Int. Cl. A61B 5/00

(52) U.S. Cl. 600/431

(57) ABSTRACT

A fiducial marker deployment system for deploying fiducial makers in targeted tissue of a living being is provided. The system includes an elongated needle, a first fiducial marker, an elongated stylet, a cartridge, and a second fiducial marker. The stylet is preloaded in the needle along with the first fiducial marker and is used to eject the first fiducial from the needle. The cartridge, which is preloaded with the second fiducial marker, can then be coupled to the needle so that the stylet can be reused to deploy the second fiducial marker, without requiring a second needle stick.

### CROSS-REFERENCE TO RELATED APPLICATIONS

This application claims priority from Provisional Application Ser. No. 60/980,926, filed on Oct. 18, 2007, entitled Fiducial Marker Deployment System Using Single Stick Needle, which application is assigned to the same assignee as this application and whose disclosure is incorporated by reference herein.

### FIELD OF THE INVENTION

This invention relates generally to fiducial markers for implantation into the body of a living being and more particularly to systems for deploying fiducial markers to keep the number of needle sticks to a minimum.

### SPECIFICATION

### BACKGROUND OF THE INVENTION

The clinical success of focused, dose-delivery procedures, such as intensity modulated radiation therapy (IMRT) and stereotactic radiation therapy (CRT), is based on the accuracy of target identification and precise patient positioning. Image-guided localization is best achieved by utilizing implanted fiducial markers. In particular, such markers allow for accurate tumor localization using a variety of visualization techniques, and help these focal radiotherapy procedures by enabling real-time targeting of tumors. In addition higher doses of radiation can be targeted to the tumor and its periphery. As the trend to higher doses, smaller number of fractions, and tighter margins becomes an important part of radiotherapy, better verifiable localization is a necessity. Image-guided localization provides this type of real-time, high-precision localization. Thus, use of such markers has been deemed imperative in situations where the targeted tissue moves with respect to external marks (e.g., tattoos). In particular, prostate, liver and other such internal organs can be much more accurately targeted using implanted markers.

The Assignee of the subject invention, CIVCO Medical Instruments, Inc., of Kalona, Iowa, offers image-guided patient localization systems for localizing targeted tissue, e.g., soft tissue, such as prostate tumors, etc., under the trademark ACCULOC®. The ACCULOC® system makes use of fiducial markers in conjunction with ISOLOC™ software and electronic portal imaging (EPID), computed radiography, or standard port films to accurately locate the tissue in which the fiducial markers are implanted and thereby provide precise patient (e.g., couch) movement to achieve desired target alignment. The markers presently sold as part of the ACCULOC® system are made of gold and are cylindrical in shape and 3 mm in length, but are available in three different diameters: 0.9 mm, 1.2 mm and 1.6 mm. The shape and size of these markers enables them to be easily inserted using a needle under ultrasound or CT guidance.

Other fiducial markers are currently available from the following companies: Best Industries, W. E. Mowrey Company, Alpha-Omega Services, Inc., and RadioMed Corporation. For example, Best Industries offers a similar marker to the markers of the ACCULOC® system, except that the Best Industries marker is not knurled. W. E. Mowrey Company, has provided markers in the form of cut-up gold wire sections of approximately 1.2×3.0 mm. Alpha-Omega Services, Inc. sells gold markers. RadioMed Corporation sells linear fiducial soft tissue markers used to localize organs, tumors and tumor beds for image-guided radiation therapy under the trade designation VISICOIL. The VISICOIL markers are in the form of an elongated helical gold coils.

The prior art also includes patents disclosing fiducial markers and method of using the same, such as U.S. Pat. No. 5,397,329 (Allen) and U.S. Pat. No. 6,694,168 (Traxel et al.).

It is a common practice to deploy such fiducial markers by means of a needle. In particular, deployment of the ACCULOC® fiducial markers is accomplished by means of a needle or cannula loaded with the fiducial markers. To that end one or more markers is disposed within the hollow central lumen in the needle just proximally of a removable plug. The plug is formed of a biologically inert sterile wax and serves to hold the marker(s) in place in the lumen of the needle between it and a pusher rod or stylet. The sharpened distal end of the needle is directed into the targeted tissue, e.g., inserted percutaneously, and the stylet actuated to push the plug and the fiducial marker out of the lumen and thereby deposit the plug and marker in the targeted tissue. If the needle contains a second marker the needle can be retracted and then introduced into a second site in the targeted tissue to deposit the second marker thereat. If the needle only contains one marker another needle will be required to deposit the second marker in the targeted tissue. In order to prevent migration the surface of each of the ACCULOC® soft tissue markers is specially treated, e.g., knurled.

If another portion of the tissue is to be tagged (as is most commonly the case), after the deployment of the marker(s) the used needle will be removed and another needle with its fiducial marker(s) and plug will be inserted into the tissue at the desired location.

As will be appreciated by those skilled in the art, the action of tagging the targeted tissue can thus involve numerous needle-sticks (e.g., each marker or marker pair deployment requiring its own needle stick). For some applications, e.g., tagging of lung tissue, it is of considerable importance to keep the number of needle sticks to an absolute minimum. The subject invention addresses that need.

### BRIEF SUMMARY OF THE INVENTION

In accordance with one aspect of this invention there is provided a fiducial marker deployment system for deploying at least two fiducial makers in the body of a living being to tag targeted tissue. The deployment system basically comprises an elongated needle, a first fiducial marker, an elongated stylet, a cartridge, and a second fiducial marker.

The elongated needle has a hub at its proximal end, sharpened tip at its distal end and a central lumen extending through the needle to the distal end. The first fiducial marker is disposed within the needle. The cartridge contains the second fiducial marker. The needle is arranged to be inserted into the targeted tissue. The stylet is arranged to be inserted into the needle to eject the first fiducial marker out of the distal end of the needle into the targeted tissue, whereupon the stylet may be removed. The cartridge is arranged to be coupled to the hub of the needle to place the second fiducial marker in communication with the central lumen of the needle, whereupon the stylet may be inserted into the cartridge and the needle and operated to eject the second fiducial marker out of the distal end of the needle.

In accordance with another aspect of this invention a method for deploying at least two fiducial makers in the body of a living being to tag targeted tissue is provided. The method basically entails providing an elongated needle, a first fiducial marker, an elongated stylet, a cartridge, and a second fiducial marker. The elongated needle has a hub at its proximal end, a sharpened tip at its distal end and a central lumen extending through it to the distal end. The first fiducial marker is disposed in the needle. The second fiducial marker is disposed in said cartridge. The needle with the first fiducial marker is inserted into the targeted tissue. The stylet is inserted into the needle to eject the first fiducial marker out of the distal end of the needle into the targeted tissue. The stylet is removed from the needle. The cartridge is coupled to the hub of the needle to place the second fiducial marker in communication with the central lumen, and the stylet is inserted into the cartridge and the needle to eject the second fiducial marker out of the distal end of the needle.

### BRIEF DESCRIPTION OF SEVERAL VIEWS OF THE DRAWINGS

FIG. 1 is a side view partially broken away showing one exemplary pre-loaded needle and its associated stylet forming a portion of the fiducial marker deployment system of this invention;

FIG. 2 is a side view, similar to FIG. 1, but showing the needle and the stylet in operation deploying a first fiducial marker from the needle;

FIG. 3 is a side view, similar to FIGS. 1 and 2, but showing the needle after it has deployed the first fiducial marker, with the stylet removed and with the cartridge containing a second fiducial marker being readied for cooperation with the needle to introduce a second fiducial marker into the needle for subsequent deployment;

FIG. 4 is a side view, similar to FIGS. 1-3, showing the now reloaded needle ready for receipt of the stylet; and

FIG. 5 is a side view, similar to FIGS. 1-4, showing the device of FIG. 4 deploying the second fiducial marker.

### DETAILED DESCRIPTION OF THE PREFERRED EMBODIMENTS OF THE INVENTION

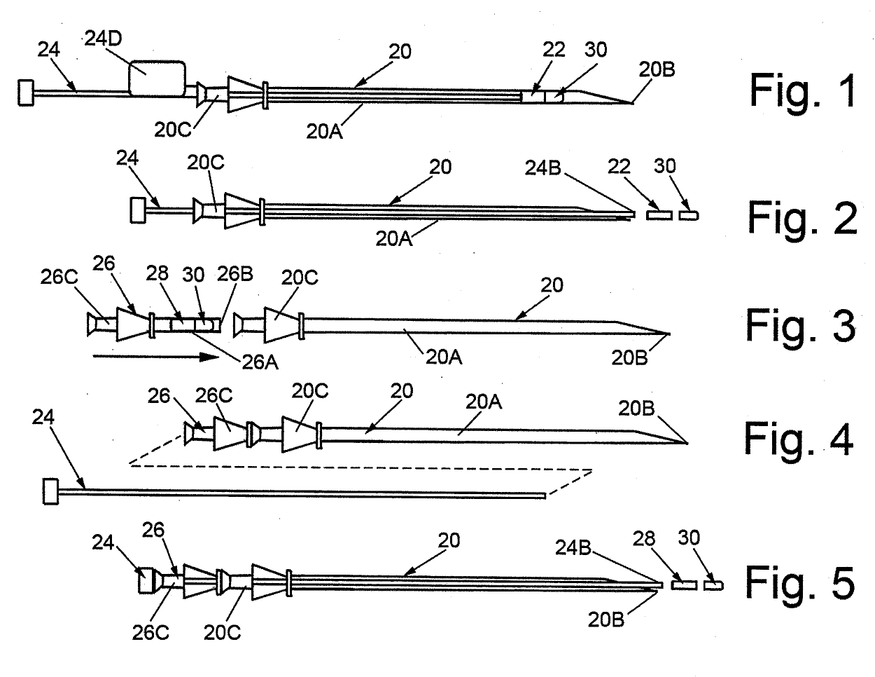

Referring now to the various figures of the drawing wherein like reference characters refer to like parts, there is shown in FIG. 1 one exemplary needle 20 pre-loaded with a first fiducial marker 22 and an associated stylet 24 forming a portion of the fiducial marker deployment system of this invention. That deployment system also includes at least one cartridge 26 containing a second fiducial marker 28. In fact, the system may include any number of cartridges so that the needle 20 of the system can be used to deploy any number of fiducial markers, all with a single needle stick.

The needle 20 is of generally conventional construction and includes a hollow body 20A formed of any suitable material, e.g., stainless steel. The free or distal end 20B of the hollow body 20A is in the form of a sharpened piercing tip. The proximal end of the body is in the form of an enlarged hub 20C. The hub can be formed of any suitable material, e.g., plastic. In the exemplary embodiment shown the needle's body is 18 ga and is 12 cm long. A central passageway or lumen extends through the entire length of the needle, i.e., through the body 20A and the hub 20C. The first fiducial marker 22 is located within the central lumen adjacent the distal free end 20B. A plug 30 is also disposed within the central lumen and is located distally of the first fiducial marker 22 to hold it in place (as is conventional). The first fiducial marker 22 (or any fiducial marker forming a portion of the subject invention) can be of any suitable construction. For example, it can be constructed in accordance with the teachings of U.S. patent application Ser. No. 11/461,813, filed on Aug. 2, 2006, entitled Fiducial Marker For Imaging Localization and Method of Using the Same, which is assigned to the same assignee as this invention and whose disclosure is incorporated by reference herein, or may be constructed in accordance with the teachings of U.S. patent application Ser. No. 11/690,470, filed on, Mar. 23, 2007 entitled Fiducial Marker With Absorbable Connecting Sleeve And Absorbable Spacer For Imaging Localization, which is also assigned to the same assignee as this invention and whose disclosure is also incorporated by reference herein. In the exemplary embodiment shown herein the fiducial markers are approximately 1.0 mm by 3.0 mm, formed of gold and having a knurled outer surface.

The stylet 24 is best seen in FIG. 4 and basically comprises an elongated body 24A having a free distal end 24B and a proximal end having a cap 24C. The stylet can be formed of any suitable material. The length of the stylet is sufficient to encompass the length of the needle and the length of the hub portion of the cartridge (as will be described later). Thus, in the exemplary embodiment of the stylet 24 shown herein it is at least 2 cm longer than the needle 20. As best seen in FIG. 1 the stylet 24 is pre-loaded, i.e., disposed in the needle 20, whereupon the elongated body 24A of the stylet extends through a portion of the central lumen of the needle 20 so that the distal free end 24B of the stylet is located just proximally of the first fiducial marker 22, while the cap 24C of the stylet is located proximally of the hub 20C of the needle. The stylet is arranged to be moved, i.e., slid longitudinally, through the central lumen of the needle to eject the plug 30 and the first fiducial marker 22 when the needle is in place within the patient's targeted tissue. This action is accomplished by pressing on the cap 24C of the stylet 24 while holding the needle in place. In order to prevent the accidental deployment (ejection) of the plug 30 and the first fiducial marker 22 the stylet 24 includes a tab 24D releasably secured to its body adjacent the cap 24C. The tab 24D can be formed of any suitable material, e.g., rubber, and is arranged to abut the proximal end of the needle's hub 20C when it is in place. This action prevents the stylet from sliding down the needle in the distal direction if accidental pressure is applied to the cap of the stylet.

The tab 24D is arranged to be removed, e.g., torn off, the stylet when operation of the stylet is desired to effect the deployment of the first fiducial marker. That operation is as follows. The needle 20 with the pre-loaded first fiducial marker 22, plug 30 and stylet 24 is introduced into to targeted tissue, e.g., percutaneously inserted into the patient's lung. When the needle's distal end 20B is at the desired position in the tissue, the tab 24D is removed, i.e., torn off. Pressure can then be applied to the cap 24D of the stylet to force the stylet to move in the distal direction through the central lumen of the needle 20. This action brings the free end 24B of the stylet into engagement with the first fiducial marker 22, whereupon continued pressure on the stylet's cap while retracting the needle slightly will result in the ejection of the plug 30 and first fiducial marker 22 from the needle into the targeted tissue as shown in FIG. 2.

After the first fiducial marker has been deployed, the system of this invention can be used to deploy a second fiducial marker in that tissue without requiring another needle stick. To that end, the heretofore identified cartridge 26 is employed. Turning now to FIG. 3, the details of the cartridge 26 will now be described. As can be seen therein the cartridge 26 is constructed similarly to the proximal portion of the needle 20. In particular, the cartridge basically comprises a tubular body section 26A and a proximally located hub 26B. A central lumen extends through the cartridge from its distal end 26C to the proximal end of the hub 26B. The outside diameter of the tubular body section 26A is just slightly less than the inside diameter of the central lumen in the hub 20B of the needle 20 to accommodate the body section 26A therein. The second fiducial marker 28 is constructed similarly to the first fiducial marker 22 and is located within the central lumen of the cartridge 26. A second plug 30 is located just distally of the second fiducial marker 28 to hold that fiducial marker in place within the cartridge.

Use of the cartridge to deploy the second fiducial marker 28 is as follows: After the first fiducial marker has been deployed as described above, the stylet 24 is removed from the needle 20, leaving the needle in place. The needle can then be repositioned to a desired location within the targeted tissues by either extending it or retracting it, without totally removing it from the targeted tissue. When the needle's distal end 20B is at its new position in targeted tissue the cartridge 26 can be brought into proximity with the hub of the needle as shown in FIG. 3 and then moved longitudinally in the direction of the arrow in that figure so that it engages the hub, e.g., the body section 26C of the cartridge is inserted into the central lumen of the hub of the needle. This action locates the second fiducial marker and the plug in the needle as shown in FIG. 4, e.g., the second fiducial marker 28 and plug 30 will be located within the lumen in the hub 20C. The stylet 24 that had been used to effect the deployment of the first fiducial marker 22 can then be reintroduced into the needle 20. In particular, the free end 24B of the stylet 24 is introduced through the central lumen in the hub 26C of the cartridge and the cap 24C of the stylet pressed while holding the needle 20 stationary. This causes the free end of the stylet to engage the second fiducial marker 28, thereby pushing it and the distally located plug 30 out of the cartridge and into the central lumen of the needle 20. Continued pressure on the stylet's cap, while retracting the needle slightly, will result in the ejection of the plug 30 and second fiducial marker 28 from the needle into the targeted tissue as shown in FIG. 5. After the second fiducial marker 28 has been deployed, the system of this invention can be used to deploy a third fiducial marker in that tissue without requiring another needle stick. To that end, a second cartridge 26 (not shown) holding a third fiducial marker and associated plug would be employed. In fact, any number of cartridges can be used to effect the deployment of any number of fiducial markers with only a single needle stick using the subject invention.

Without further elaboration the foregoing will so fully illustrate my invention that others may, by applying current or future knowledge, adopt the same for use under various conditions of service.

What is claimed is:

1. A fiducial marker deployment system for deploying at least two fiducial makers in the body of a living being to tag targeted tissue, said system comprising an elongated needle, a first fiducial marker, an elongated stylet, a cartridge, and a second fiducial marker, said elongated needle having a hub at its proximal end, a sharpened tip at its distal end and a central lumen extending through said needle to said distal end, said first fiducial marker being disposed in said needle, said cartridge containing said second fiducial marker, said needle being arranged to be inserted into the targeted tissue, said stylet being arranged to be inserted into said needle to eject said first fiducial marker out of said distal end of said needle into the targeted tissue, whereupon said stylet may be removed, said cartridge being arranged to be coupled to said hub of said needle to place said second fiducial marker in communication with said central lumen, whereupon said stylet may be inserted into said cartridge and said needle to eject said second fiducial marker out of said distal end of said needle.

2. The fiducial marker deployment system of claim 1 wherein said cartridge comprises a tubular body section having a central lumen in which said second fiducial marker is located.

3. The fiducial marker deployment system of claim 1 additionally comprising a plug located within said central lumen distally of said first fiducial marker.

4. The fiducial marker deployment system of claim 2 additionally comprising a first and second plugs, said first plug being located within said central lumen of said needle distally of said first fiducial marker, said second plug being located in said central lumen of said cartridge distally of said second fiducial marker.

5. The fiducial marker deployment system of claim 1 wherein said fiducial marker has a knurled outer surface.

6. The fiducial marker deployment system of claim 1 wherein said stylet comprises an elongated body having a free distal end and a proximal end having a cap, said stylet being pre-loaded in said needle, whereupon said elongated body extends through a portion of said central lumen of said needle so that said distal free end of said stylet is located proximally of said first fiducial marker and said cap of said stylet is located proximally of said hub of said needle.

7. The fiducial marker deployment system of claim 6 wherein said elongated body of said stylet is arranged to be slid down said central lumen when a force directed to said cap.

8. The fiducial marker deployment system of claim 7 wherein stylet includes a member releasably secured to said elongated body of said stylet adjacent said cap to prevent the accidental sliding of said elongated body of said stylet.

9. The fiducial marker deployment system of claim 7 wherein said member comprises a rubber tab.

10. A method for deploying at least two fiducial makers in the body of a living being to tag targeted tissue, said method comprising:
providing an elongated needle, a first fiducial marker, an elongated stylet, a cartridge, and a second fiducial marker, said elongated needle having a hub at its proximal end, a sharpened tip at its distal end and a central lumen extending through said needle to said distal end, said first fiducial marker being disposed in said needle, said second fiducial marker being disposed in said cartridge,
inserting said needle with said first fiducial marker therein into the targeted tissue,
inserting said stylet into said needle to eject said first fiducial marker out of said distal end of said needle into the targeted tissue,
removing said stylet from said needle,
coupling said cartridge to said hub of said needle to place said second fiducial marker in communication with said central lumen, and
inserting said stylet into said cartridge and said needle to eject said second fiducial marker out of said distal end of said needle.

11. The method of claim 10 wherein said cartridge comprises a tubular body section having a central lumen in which said second fiducial marker is located.

12. The method of claim 10 additionally comprising providing a plug within said central lumen distally of said first fiducial marker.

13. The method of claim 10 comprising providing comprising a first and second plugs, said first plug being located within said central lumen of said needle distally of said first fiducial marker, said second plug being located in said central lumen of said cartridge distally of said second fiducial marker.

14. The method of claim 10 wherein said stylet comprises an elongated body having a free distal end and a proximal end having a cap, and wherein said method comprising pre-loading said stylet in said needle, whereupon said elongated body extends through a portion of said central lumen of said needle so that said distal free end of said stylet is located proximally of said first fiducial marker and said cap of said stylet is located proximally of said hub of said needle.

15. The method of claim 14 wherein said method comprises providing a force on said cap to cause said elongated body of said stylet to slide down said central lumen.

16. The method of claim 15 wherein said method additionally comprises providing a member releasably secured to said elongated body of said stylet adjacent said cap to prevent the accidental sliding of said elongated body of said stylet.

17. The method of claim 16 wherein said member comprises a rubber tab.
